(19) United States
(12) Patent Application Publication
(10) Pub. No.: US 2024/0050224 A1
(43) Pub. Date: Feb. 15, 2024

(54) INTRAOCULAR LENS DELIVERY DEVICES AND METHODS OF USE

(71) Applicant: Alcon Inc., Fribourg (CH)

(72) Inventors: Daniel HILDEBRAND, Santa Cruz, CA (US); Claudio ARGENTO, Felton, CA (US); Gregory MATTHEWS, San Francisco, CA (US)

(73) Assignee: Alcon Inc., Fribourg (CH)

(21) Appl. No.: 18/458,061

(22) Filed: Aug. 29, 2023

Related U.S. Application Data
(63) Continuation of application No. 16/777,698, filed on Jan. 30, 2020, now Pat. No. 11,779,456.
Application No. 16/777,698 is a continuation of application No. 15/635,080, filed on Jun. 27, 2017, now Pat. No. 10,595,989.
Application No. 15/635,080 is a continuation of application No. 14/728,824, filed on Jun. 2, 2015, now Pat. No. 9,693,858.
Application No. 14/728,824 is a continuation of application No. 14/163,794, filed on Jan. 24, 2014, now Pat. No. 9,044,317.
Application No. 14/163,794 is a continuation of application No. 13/180,427, filed on Jul. 11, 2011, now Pat. No. 8,668,734.
(60) Provisional application No. 61/363,155, filed on Jul. 9, 2010.

(51) Int. Cl. A61F 2/16

(52) U.S. Cl. CPC A61F 2/1678; A61F 2/1672; A61F 2/167; A61F 2/1675; A61F 2/1691; A61F 2002/16903; A61F 2002/1681; A61F 2002/16905

(57) ABSTRACT

Intraocular lens delivery devices and methods of use.

### CROSS-REFERENCE TO RELATED APPLICATIONS

This application is a continuation of U.S. application Ser. No. 16/777,698, filed Jan. 30, 2020, which is a continuation of U.S. application Ser. No. 15/635,080, filed Jun. 27, 2017, now U.S. Pat. No. 10,595,989, which is a continuation of U.S. application Ser. No. 14/728,824, filed Jun. 2, 2015, now U.S. Pat. No. 9,693,858, which is a continuation of U.S. application Ser. No. 14/163,794, filed Jan. 24, 2014, now U.S. Pat. No. 9,044,317, which is a continuation of U.S. application Ser. No. 13/180,427, filed Jul. 11, 2011, now U.S. Pat. No. 8,668,734, which application claims the benefit of U.S. Provisional Application No. 61/363,155, filed Jul. 9, 2010, all of which are incorporated herein by reference in their entirety.

### BACKGROUND

Intraocular lens delivery systems have been developed but have several shortcomings. To implant an intraocular lens into an eye, an incision needs to be made in the eye to allow for passage of a delivery device and/or the intraocular lens. In general, it is desirable to make the incision is small as possible to cause the least amount of damage to the eye and shorten the healing process. Depending on the configuration and/or size of the intraocular lens, some intraocular lenses need to be deformed, sometimes controllably, into a smaller delivery profile so they will fit through the incision and into the eye. Delivery devices and/or systems are needed that can controllably deform or alter the configuration or orientation of the intraocular lens for delivery through an incision in an eye.

### SUMMARY OF THE DISCLOSURE

One aspect of the disclosure is an intraocular lens delivery system, comprising a delivery device with a port therein adapted to receive a cartridge; and a cartridge adapted to receive an intraocular lens therein, the cartridge having a leading haptic receiving area and a trailing haptic receiving area. The port can be a side port, and the delivery device can further comprise a positioning element to align the cartridge and delivery device at a predetermined position.

In some embodiments the cartridge comprises a channel adapted to be in communication with a channel within the delivery device. The cartridge can include an optic receiving area, and wherein the delivery device channel, the cartridge channel, and the optic receiving area are all in fluid communication.

In some embodiments the delivery device includes a first actuator adapted to load an intraocular lens into a distal loading portion of the delivery device, and a second actuator adapted to control a second delivery element to deploy the intraocular lens from the distal loading portion.

One aspect of the disclosure is a cartridge adapted to receive an intraocular lens therein, comprising a first portion with an optic receiving area, a leading haptic receiving area, and a trailing haptic receiving area; and a second portion coupled to the first portion and adapted to be in open and closed positions relative to the first portion.

In some embodiments the second portion has a complimentary optic portion receiving area, a leading haptic receiving area, and a trailing haptic receiving area. The first portion and second portion can be coupled at a hinge. In some embodiments the leading haptic receiving area is distal to the optic receiving area. In some embodiments the trailing haptic receiving area is disposed to the side of the optic receiving area. In some embodiments the cartridge further comprises a channel in communication with the optic receiving area. The channel can be disposed proximal to the optic receiving area.

One aspect of the disclosure is a delivery device adapted to deliver an intraocular lens, comprising an elongate body with a lumen therein; a plunger disposed within the elongate body and adapted to move axially with respect to the elongate body; and an actuator adapted to prime a fluid delivery assembly and dispense a fluid through a fluid channel within the elongate body.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIGS. 1A-1C illustrate an exemplary delivery system including a delivery device and cartridge.

FIGS. 2A-2C illustrate an exemplary cartridge and intraocular lens.

FIGS. 3A-3E illustrate an exemplary method of delivering an intraocular lens using the delivery device and cartridge.

### DETAILED DESCRIPTION

One aspect of the disclosure is a delivery system including a delivery device adapted to receive a cartridge therein. The cartridge is adapted to receive an intraocular lens. In use, the delivery system delivers the intraocular lens from the cartridge and the delivery device and into an eye.

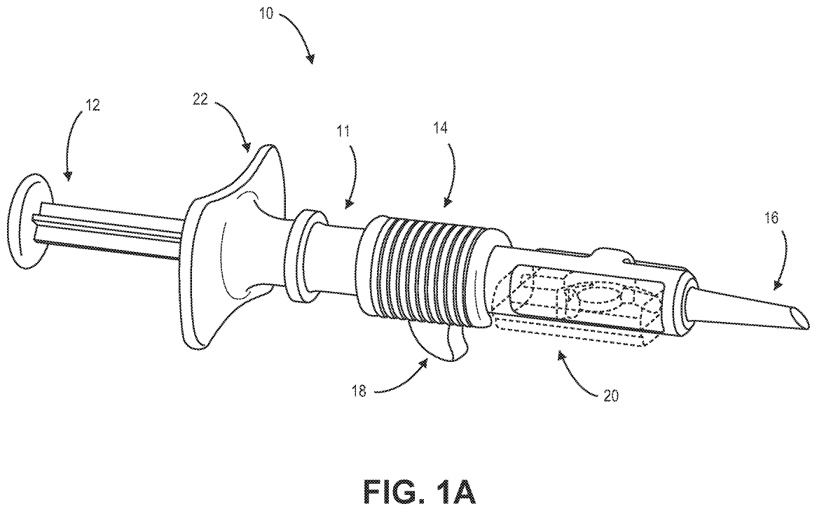

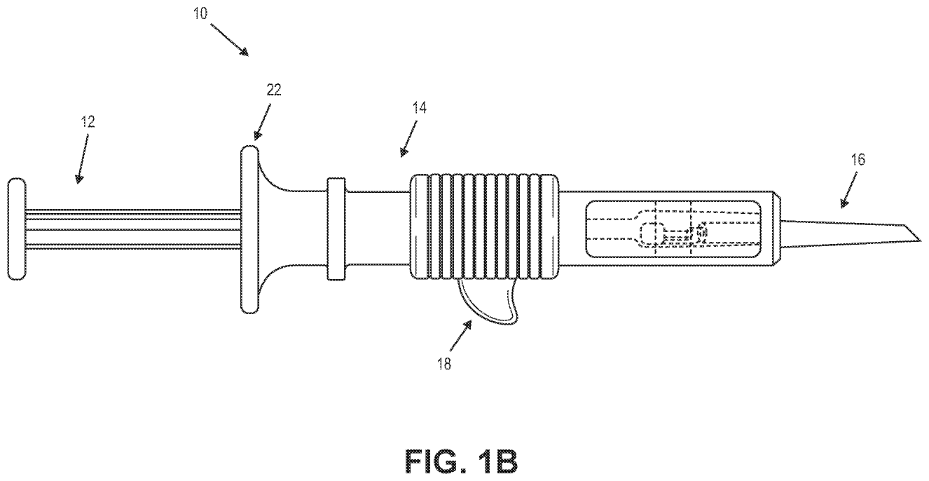

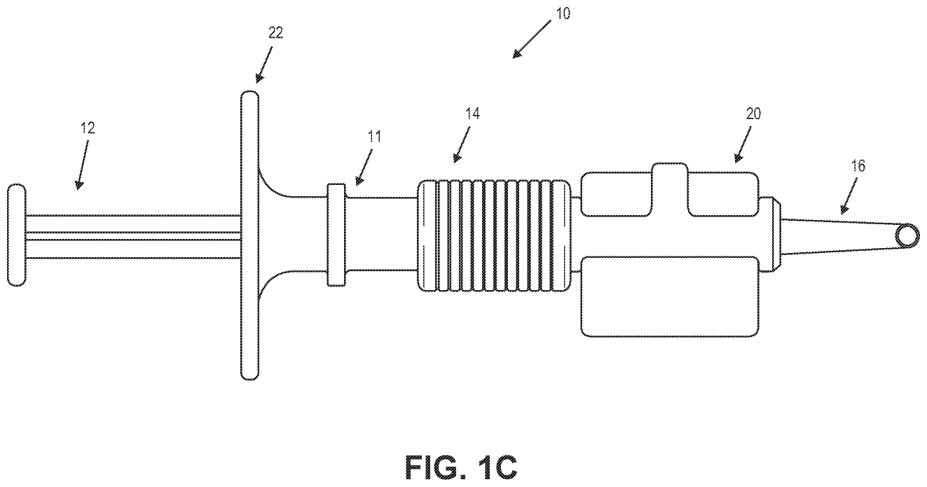

FIGS. 1A-1C show an exemplary system including delivery device 10 and cartridge 20 disposed therein. An intraocular lens is not show in the cartridge for clarity, and a variety of intraocular lenses could be used with the systems herein. Delivery device 10 includes elongate body 11 extending between proximal portion 22 and distal portion 16. Elongate body 11 is adapted to receive plunger 12 therein, such that device functions similarly to a syringe. Elongate body 11 includes a port extending therethrough, adapted to receive cartridge 20. Device 10 also includes first actuator 14 and second actuator 18, the latter in the form of a trigger. Distal portion 16 is in the form of a nozzle with a distal port from which the intraocular lens is deployed. In this embodiment the distal port has a beveled end. Distal portion 16 is also tapered towards the distal end. The cross section at the distal end is generally elliptical. FIGS. 1B and 1C show side view and bottom views, respectively, of cartridge 20 disposed within device 10.

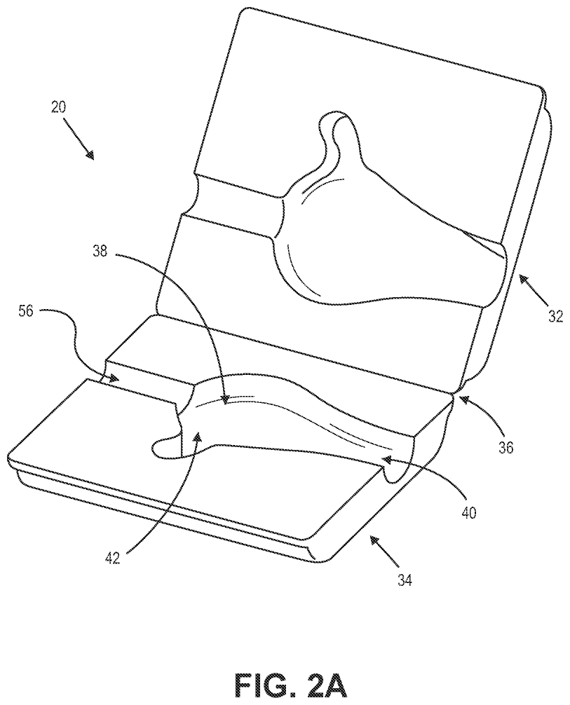

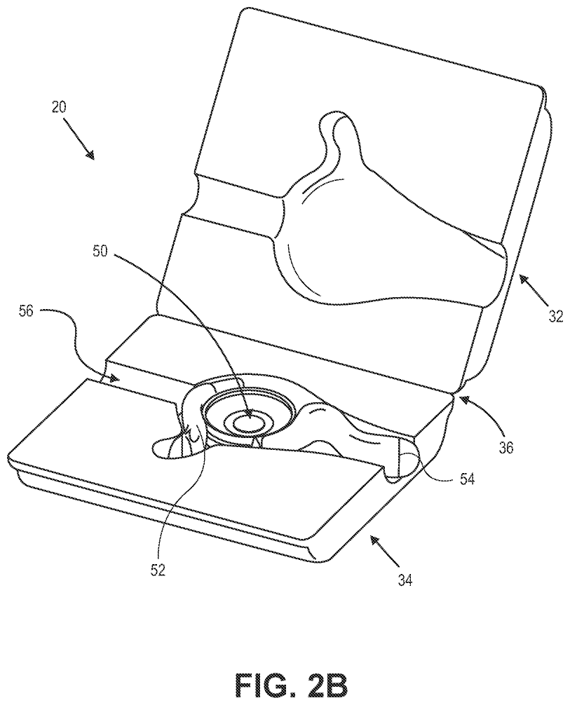

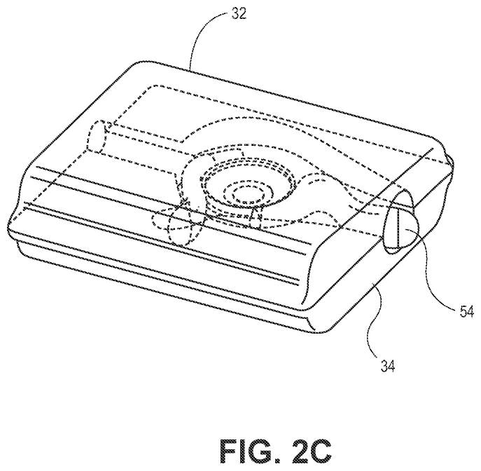

FIG. 2A illustrates cartridge 20 in an open configuration and adapted to receive an intraocular lens therein. Cartridge 20 is hinged with a first portion 32 and a second portion 34 coupled by any type of hinge 36 (e.g., a living hinge). Cartridge 20 has been formed with an intraocular lens receiving area, which includes an optic portion receiving area 38, leading haptic receiving area 40, and trailing haptic receiving area 42. In some embodiments the cartridge is molded with these areas formed therein. Cartridge also includes channel 56 disposed proximal to the lens receiving area, adapted to receive a portion of delivery system, a fluid, etc., therethrough.

FIG. 2B shows an exemplary intraocular lens disposed within cartridge 20. In this embodiment, intraocular lens includes an optic portion 50 and haptics 52 and 54. Optic 50 has been positioned within optic receiving area 38, haptic 54 has been positioned within leading haptic receiving area 40, and haptic 52 has been positioned within trailing haptic receiving area 42. Haptic 54 is disposed distal to the optic 50, while haptic 52 is positioned generally to the side of optic 50, but could be disposed in any position relative to the optic. For example, haptic 52 could be positioned in a more trailing position if haptic receiving area 42 were formed in such a position.

In this exemplary embodiment, the intraocular lens is a fluid-driven intraocular lens, such as is described in US 2008/0306588, filed Jul. 22, 2008, now U.S. Pat. No. 8,328,869, the disclosure of which is incorporated by reference herein. Optic portion 50 is in fluid communication with haptics 52 and 54. Fluid distribution during delivery and other methods of use that can be incorporated into the systems herein can be found in US 2009/0030425, filed Jul. 23, 2008, the disclosure of which is incorporated by reference herein.

In FIG. 2C, first portion 32 has been closed relative to second portion 34 such that the cartridge is in a closed position, ready to be positioned within delivery device 10. When portion 32 is closed, some of the fluid within the lens may be slightly redistributed within the lens, or it may not be.

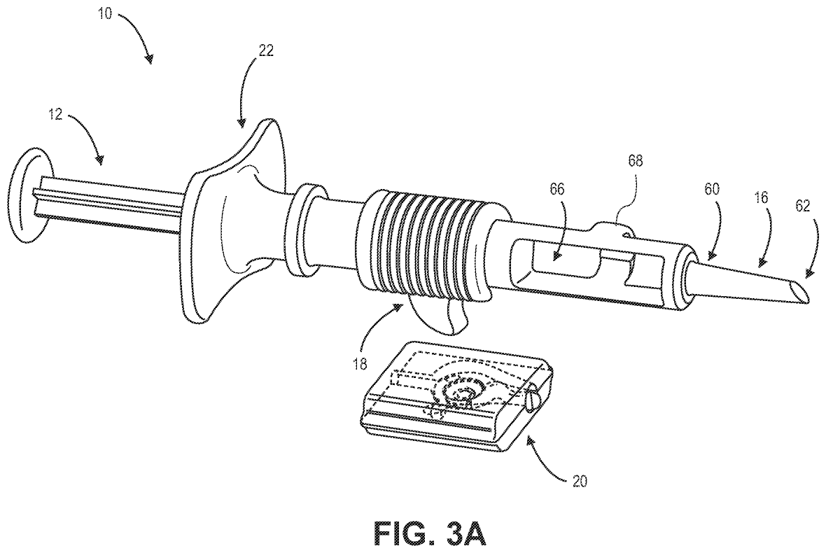

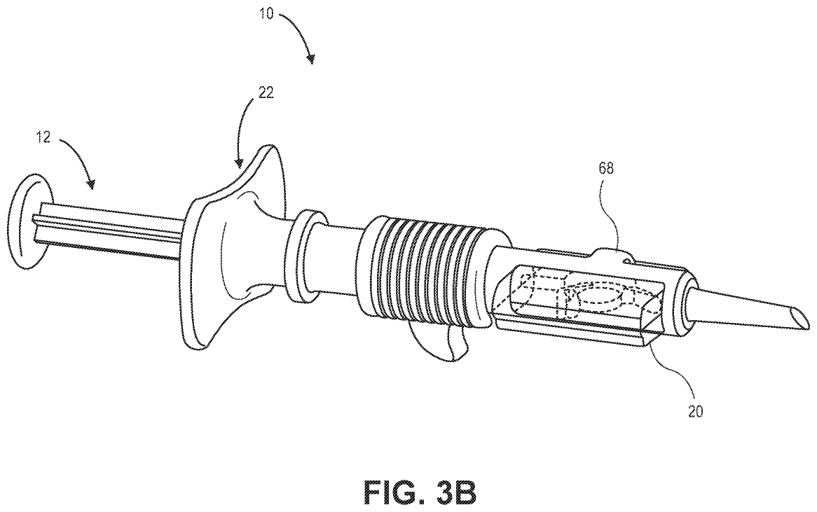

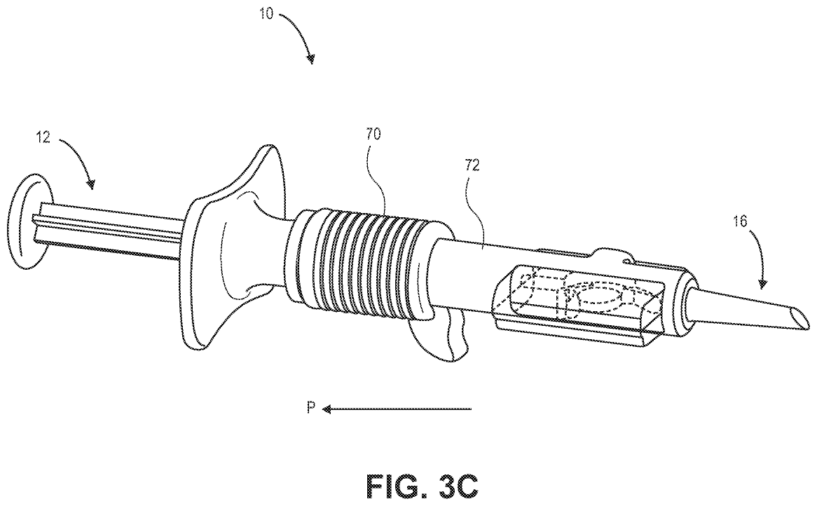

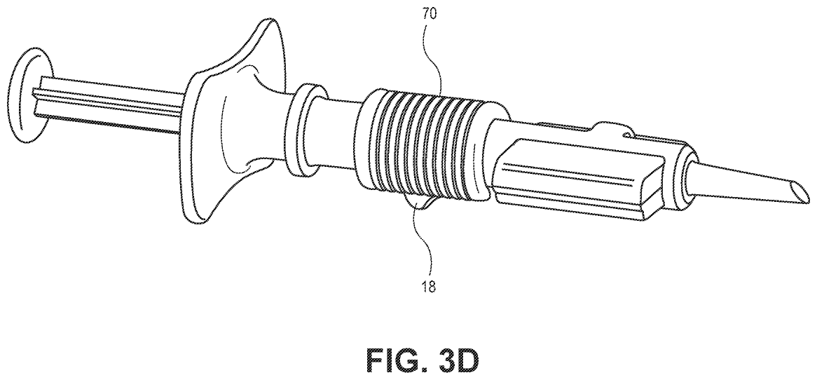

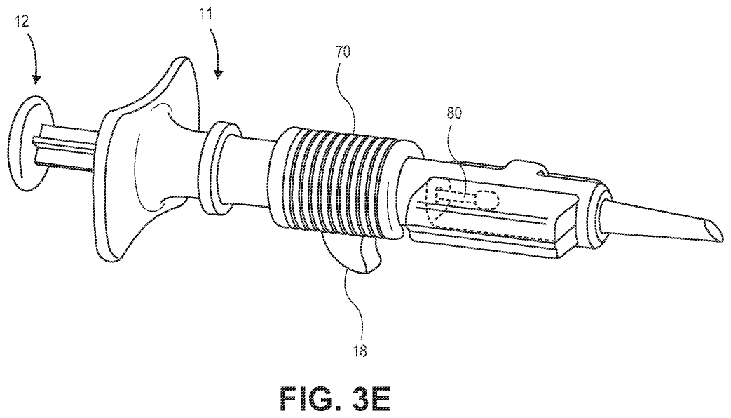

FIG. 3A illustrates cartridge 20 with an intraocular lens disposed therein, and delivery device 10, including port 66 adapted to receive cartridge 20 therethrough. Device 10 also includes positioning element 68 adapted to maintain cartridge 20 at a predetermined position with respect to port 66. The positioning element 68 is positioned to align a channel within device 10 with channel 56 in cartridge 20. This allows a fluid, a portion of delivery device, etc., to be advanced through the delivery device and the cartridge to deploy the intraocular lens, as described below.

FIG. 3B shows cartridge 20 advanced through port 66 (which could also be a top port or bottom port) and engaging positioning element 68.

Actuator 70 is then retracted in the proximal direction (“P”), which activates, or primes, fluid delivery. In FIG. 3D, actuator 18 in the form of a trigger is actuated, causing actuator 70 to be advanced forward, moving a fluid (or mechanical component) through channel 56 in cartridge 20. This causes the intraocular lens to be advanced through cartridge and loaded into the nozzle of device 10. As is it being loaded, the haptics and/or optic are deformed into a delivery configuration. Positioning the haptics as described with respect to FIGS. 2A-2C, the leading haptic is first loaded, followed by the optic, and then by the trailing haptic. This configuration eases the loading on the lens. While actuation of actuator 18 can move a fluid through the system to load the lens, actuating actuator 18 can alternatively or in addition to move a piston or plunger component that engages and pushes the lens into the nozzle. The plunger can be adapted to engage the optic, bypassing the trailing haptic.

In FIG. 3E, plunger 12 is advanced with respect to body 11 and within body 11, causing plunger 80 to be advanced relative to body 11 and to the intraocular lens. This plunger is advanced until it engages with the lens and drives it out of the nozzle and into an eye.

Intraocular lenses with different configurations can be delivered with the cartridges and delivery devices herein, as long as the cartridge is adapted to receive them. For example, the cartridge may need to be molded in a different way to receive a different intraocular lens.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A method of delivering a fluid-driven intraocular lens, comprising:
coupling a cartridge housing the fluid-driven intraocular lens to a delivery device, wherein the cartridge comprises an optic portion receiving area, a leading haptic receiving area, and a trailing haptic receiving area; and
activating a first actuator to cause a leading haptic of the fluid-driven intraocular lens to move from the leading haptic receiving area and to advance the fluid-driven intraocular lens distally out of the cartridge.

2. The method of claim 1, wherein the trailing haptic receiving area extends laterally away from an axis passing through the optic portion receiving area.

3. The method of claim 1, wherein activating the first actuator further causes a trailing haptic of the fluid-driven intraocular lens to be deformed to a straightened configuration as the trailing haptic moves from the trailing haptic receiving area.

4. The method of claim 1, wherein activating the first actuator further causes a trailing haptic of the fluid-driven intraocular lens to move from the trailing haptic receiving area.

5. The method of claim 4, wherein when the trailing haptic is positioned in the trailing haptic receiving area, a segment of a trailing haptic of the fluid-driven intraocular lens curves circumferentially around an optic portion of the fluid-driven intraocular lens.

6. The method of claim 5, wherein the leading haptic and the trailing haptic are in fluid communication with the optic portion of the fluid-driven intraocular lens.

7. The method of claim 6, wherein a terminal end of the leading haptic is configured to point away from the optic portion of the fluid-driven intraocular lens when the fluid-driven intraocular lens is moved out of the cartridge.

8. The method of claim 1, wherein the cartridge has an open configuration and a closed configuration, and wherein the cartridge is coupled to the delivery device in the closed configuration.

9. The method of claim 8, wherein part of the delivery device is proximal to the cartridge when the cartridge is coupled to the delivery device and another part of the delivery device is distal to the cartridge.

10. The method of claim 1, wherein the fluid-driven intraocular lens is advanced distally out of the cartridge into a nozzle of the delivery device.

11. The method of claim 10, further comprising activating a second actuator to cause the fluid-driven intraocular lens to advance out of the nozzle.

12. A system for delivering an intraocular lens, comprising:
a cartridge configured to house the intraocular lens, the cartridge comprising:
a first channel,
an optic receiving area,
a leading haptic receiving area, and
a trailing haptic receiving area extending laterally away from an axis passing through the first channel and the optic receiving area; and
a delivery device, comprising:
a port adapted to receive the cartridge,
a second channel configured to be aligned with the first channel when the cartridge is received at the port of the delivery device, and
an actuator configured to advance the intraocular lens through the cartridge.

13. The system of claim 12, wherein the delivery device further comprises a positioning element to facilitate alignment of the first channel with the second channel when the cartridge is inserted into the port of the delivery device.

14. The system of claim 12, wherein the intraocular lens is advanced through the cartridge and into a nozzle of the delivery device.

15. The system of claim 12, wherein the actuator is configured to advance a fluid through the cartridge.

16. The system of claim 15, wherein the first channel and the second channel are in fluid communication.

17. The system of claim 12, wherein the leading haptic receiving area is distal to the optic receiving area.

18. The system of claim 12, wherein the cartridge has an open configuration and a closed configuration, and wherein the cartridge is coupled to the delivery device in the closed configuration.

19. The system of claim 18, wherein part of the delivery device is proximal to the cartridge when the cartridge is coupled to the delivery device and another part of the delivery device is distal to the cartridge.

20. The system of claim 18, wherein the cartridge comprises a first portion and a second portion connected by a hinge, wherein the cartridge is configured to redistribute fluid within the intraocular lens when the intraocular lens is housed within the cartridge and the cartridge is in the closed configuration.
